(19) United States
(12) Patent Application Publication
(10) Pub. No.: US 2012/0308652 A1
(43) Pub. Date: Dec. 6, 2012

(54) ORAL FORM OF ADMINISTRATION COMPRISING ENTECAVIR

(76) Inventors: Daniela Stumm, Berlin (DE); Jana Paetz, Bonn (DE)

(21) Appl. No.: 13/517,981

(22) PCT Filed: Dec. 22, 2010
(86) PCT No.: PCT/EP10/07892

§ 371 Date: Aug. 7, 2012

(30) Foreign Application Priority Data
Dec. 23, 2009 (DE) 10-2009-060-194.5
Oct. 15, 2010 (DE) 10-2010-048.635.3

(51) Int. Cl. A61K 31/522; A61P 31/14; C07D 473/18; A61K 9/48

(52) U.S. Cl. 424/451; 514/263.37; 544/276

(57) ABSTRACT

The invention relates to pharmaceutical formulations, preferably in the form of an oral dosage form, for the treatment of chronic hepatitis-B virus infections, containing micronised Entecavir, and processes for preparing it. The invention further relates to intermediates containing micronised entecavir, in which the D50 value for the particle size distribution is less than 50 μm, and processes for preparing them.

The invention relates to pharmaceutical formulations, preferably in the form of an oral dosage form, for the treatment of chronic hepatitis B virus infections, containing micronised entecavir, and processes for preparing it. The invention further relates to intermediates containing micronised entecavir, in which the D50 value for the particle size distribution is less than 50 μm, and processes for preparing them.

Entecavir is a virostatic agent which is approved for the treatment of chronic hepatitis B virus infections. It belongs to the group of nucleoside reverse transcriptase inhibitors (NRTI) and is a chemical analogue of the nucleoside guanosine. Entecavir selectively inhibits HBV polymerase and thus the synthesis of DNA and the replication of the hepatitis B virus in infected cells.

Hepatitis B viruses are viruses with cubic capsid symmetry, which contain an annular double-stranded DNA. Their surface is formed by the hepatitis B surface antigen (HBsAg). The core contains the core antigen HBcAg and its cleavage product HBeAg, and also a DNA polymerase and a phosphokinase. Free HBcAg can only be detected in liver cell nuclei. HBeAg can be detected in the blood in the acute stage of hepatitis and in some chronic virus carriers and, like HbsAg, is regarded as an important indicator of infectiousness. In certain mutations of the core or precore gene, no HBeAg is formed; in this case, assays for HbeAg lead to a false negative assessment of the active virus replication in those infected with HBV.

The IUPAC name of entecavir is (1S,3R,4S)-2-amino-1,9-dihydro-9-[4-hydroxy-3-hydroxymethyl-2-methylene cyclopentyl]-6H-purine-6-on. The chemical structure of entecavir is shown in formula (1) below:

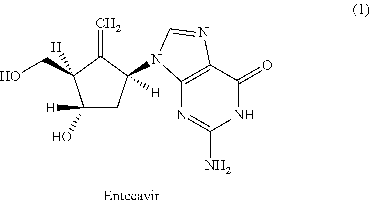

The synthesis of entecavir was described in WO 98/09964. Pharmaceutical compositions containing entecavir are known from WO 2001/64221.

Entecavir is marketed under the trade name Baraclude® as a film-coated tablet or oral solution. In the film-coated tablet, the content of entecavir as the active agent based on the total weight of the film-coated tablet is very small—in the standard dosages, it is 0.5 or 1 mg entecavir, based on a total weight of the film-coated tablet of more than 200 mg, which is less than 0.5% by weight. This small content of active agent can lead to considerable problems during the manufacture of the formulation with regard to the uniformity of the content. For example, minor changes in the content of active agent, caused by changes in the flowability, or separation tendencies can lead to variations.

Ph. Eur. 6.0 section 2.9.6 there lays down a content uniformity test, in which each individual content of 10 units must lie between 85 and 115 percent of the average content. If more than one individual content lies outside that limit or if one individual content lies outside the limits of 75 to 125 percent of the average content, the test is not passed. According to WO 2001/64221, the necessary uniformity of the content cannot be ensured simply by mixing the ingredients or by employing conventional granulation methods.

An inhomogeneous distribution of the active agent during production of the formulation can result both in too low and also too high a content of the active agent entecavir in the dosage form, e.g. a film-coated tablet. This in turn leads to an underdose or overdose in the patient. Especially in the case of entecavir, however, accurate dosing of the active agent is indispensable, because if the dosage is too high, substantial symptoms of poisoning may occur. If the dose is too low, on the other hand, the plasma level and hence the efficacy may be reduced so drastically that the therapeutic success may be jeopardised.

The problem of the present invention was therefore to overcome the above-mentioned disadvantages. The intention is to provide an oral dosage form containing entecavir, which causes neither an underdose nor an overdose in the patient when used correctly. In addition, the intention is to provide an oral dosage form containing entecavir which ensures advantageous bioavailability in the patient. A further problem of the present invention consists in providing an oral dosage form containing entecavir whose content of active agent, even after a lengthy storage time, lies within the concentration limits of 85 and 115 percent of the average content according to Ph. Eur. In contrast to WO 2001/064221, the problems are also supposed to be soluble using a smaller amount of apparatus, i.e. by means of conventional mixing or granulation technology.

It has unexpectedly been possible to solve the above-mentioned problems by jointly grinding, especially jointly micronising, entecavir (a) and excipients (b), preferably with a specific selection of excipients. In the context of this application, the expression “excipients (b)” generally refers to one or more excipient(s).

One subject matter of the invention is therefore an intermediate comprising (a) entecavir and (b) excipients, in which the D50 value for the particle size distribution of the intermediate is less than 50 μm.

A further subject matter of the invention is an oral dosage form, preferably a solid oral dosage form, containing the intermediate of the invention.

A further subject matter of the invention is a process for preparing an intermediate comprising (a) entecavir and (b) excipients, in which the D50 value for the particle size distribution of the intermediate is less than 50 μm, comprising the steps of.
(i) mixing (a) entecavir and (b) excipients, and
(ii) grinding (a) entecavir and (b) excipients.

The subject matter of the invention is also a process for producing an oral dosage form conaining (a) entecavir and (b) excipients, comprising the steps of:
(i) mixing (a) entecavir and (b) excipients, and
(ii) grinding (a) entecavir and (b) excipients to produce an intermediate in which the D50 value for the particle size distribution of the intermediate is less than 50 μm,
(iii) optionally granulating the intermediate
(iv) pressing the intermediate from step (ii) or (iii) or filling the intermediate from step (iii) into dosage forms such as sachets or capsules,
   wherein further pharmaceutical excipients may optionally be added before or during steps (iii) and (iv).

The subject matter of the invention is also micronised entecavir.

Finally, one subject matter of the invention is micronised entecavir for the treatment of chronic hepatitis B virus infections, especially for treating HBeAg-negative patients. In the context of this invention, the term “entecavir” comprises (1S,3R,4S)-2-amino-1,9-dihydro-9[4-hydroxy-3-hydroxymethyl-2-methylene cyclopentyl]-6H-purine-6-on in accordance with formula (I) above. In addition, the term “entecavir” comprises all the pharmaceutically acceptable salts, hydrates and/or solvates thereof. For all the embodiments of this invention, the term “entecavir” preferably means entecavir in crystalline form, i.e. preferably more than 90% by weight of the entecavir used is present in crystalline form, especially 100% by weight. In the context of this invention, entecavir (a) is preferably used as the sole active agent.

In a preferred embodiment, the entecavir (a) used, or alternatively its pharmaceutically acceptable salt, has a water content of 0.01 to 10% by weight, more preferably 4.0 to 8.0% by weight, even more preferably 5.0 to 7.0% by weight, particularly preferably 5.5 to 6.5% by weight. In the context of this application, the water content is preferably determined according to the Karl Fischer method, using a coulometer at 160° C. A Metrohm 831 KF coulometer with a titration cell without a diaphragm is preferably used. Usually, a 20 mg sample of entecavir is analysed.

“Micronised entecavir” is understood, according to the invention to mean particulate entecavir with a D50 value for the particle size distribution of 0.01 to 50 μm, preferably 0.1 to 30 μm, more preferably 1 to 20 μm, particularly preferably 1.5 to 15 μm and especially 2 to 10 μm. Micronised entecavir is usually obtainable by grinding or milling, preferably in the following milling apparatuses: ball mill, air jet mill, pin mill, classifier mill, cross beater mill, disk mill, edge mill, mortar grinder, rotor mill, roller crusher, hammer mill. It is preferable to use a peg mill, e.g. a Netsch MicroCer.

Entecavir is generally used in amounts between 0.1 and 10% by weight, preferably between 0.15 and 5.0% by weight, particularly preferably between 0.2 and 2.0% by weight, based on the total weight of the intermediate of the invention.

In the context of the invention, entecavir (a) together with excipients (b) forms an intermediate in which the D50 value for the particle size distribution of the intermediate is less than 50 μm.

According to the present invention, an “intermediate” is usually understood to mean a pharmaceutical composition which is not administered directly, but is instead converted into an applicable oral dosage form by means of suitable processes, such as granulation and/or compression.

When the “particle size” of a particle is to be determined, this is understood for the purposes of the invention to mean the diameter of an equivalent particle which is assumed to be spherical and to have the same light-scattering pattern as the particles to be analysed. In accordance with the invention, the particle size is determined by means of laser diffractometry. Specifically, a Malvern Instruments Mastersizer 2000 was used to determine the particle diameter. Wet measurement with dispersion in dispersant, 2,000 rpm ultrasound for 30 sec. is preferred. The evaluation is carried out for particles with a D50 value of less than 5.0 μm using the Mie method and for particles with a D50 value of 5.0 μm or more using the Fraunhofer method.

“Particle size distribution of the intermediate” is to be understood in the context of this invention as meaning the statistical distribution of the volume portions based on all the particle sizes of the particles of the intermediate. “Volume portion” according to the invention means the volume-based proportion in percent of all particles with a defined particle size.

According to the invention, the D50 value for the particle size distribution describes the particle size at which 50% by volume of the particles have a smaller particle size than the particle size corresponding to the D50 value. Likewise, 50% by volume of the particles then have a larger particle size than the D50 value.

The D90 value for the particle size distribution of the intermediate is accordingly defined as the particle size at which 90% by volume of the particles have a smaller particle size than the particle size corresponding to the D90 value.

Analogously, the D10 value for the particle size distribution of the intermediate is defined as the particle size at which 10% by volume of the particles have a smaller particle size than the particle size corresponding to the D10 value.

In the context of the invention, the intermediate has a D50 value for the particle size distribution of 0.1 to 50 μm, preferably 0.1 to 30 μm, more preferably 1 to 20 μm, particularly preferably 1.5 to 15 μm and especially 2 to 10 μm.

In a preferred embodiment, the intermediate usually has a D10 value for the particle size distribution of between 4 and 12 μm, preferably between 5 and 10 μm. In an alternative embodiment, the intermediate has a D10 value between 0.05 and 5 μm, preferably between 1.0 and 4.5 μm auf. The expression “between 4 and 12 μm” (for example) is synonymous here with the expression “from 4 to 12 μm”.

In a further preferred embodiment, the intermediate usually has a D90 value for the particle size distribution of less than 250 μm, preferably less than 200 μm, more preferably 10 to 180 μm, even more preferably 15 to 160 μm, especially 20 to 120 μm.

In a further preferred embodiment, the ratio between the D90 value and D50 value (=D90/D50) has a value of between 1 and 10, preferably between 2 and 7. In an alternative embodiment, the ratio has a value of between 1.1 and 5.0, preferably between 1.2 and 3.0, particularly preferably between 1.3 and 2.5.

In a further preferred embodiment, the ratio between the D50 value and D10 value (=D50/D10) has a value of between 1 and 10, preferably between 2 and 7. In an alternative embodiment, the ratio has a value of between 1.1 and 5.0, preferably between 1.2 and 3.0, particularly preferably between 1.3 and 2.5.

In a further preferred embodiment, the ratio between the D90 value and D10 value (=D90/D10) has a value of between 5 and 20, preferably between 7 and 17. In an alternative embodiment, the ratio has a value of between 1.1 and 5.0, preferably between 6.0 and 3.0, particularly preferably between 1.3 and 1.5.

The particle size distribution of the intermediate of the invention can be monomodal or bimodal. In a preferred embodiment of the invention, the particle size distribution of the intermediate is monomodal. “Monomodal” is in this case understood to mean that the particle size distribution only has one maximum when represented in a histogram and/or a frequency distribution curve.

In the context of the invention, entecavir (a) together with excipients (b) forms an intermediate.

In a preferred embodiment, the term “excipients” comprises fillers (b₁), surface stabilisers (b₂), disintegrants (b₃), flow conditioning agents (b₄) and/or lubricants (b₅). Where appropriate, wetting agents (b₆) can also be used as excipients.

For the purposes of this invention, fillers (b₁) are understood to mean substances which are described as pharmaceutical fillers in the state of the art. These fillers are typically substances which are needed in order to form the body of the oral dosage form in the case of dosage forms with small amounts of active agent, so as to obtain a sufficient amount of dosage form mixture for a suitable dosage form size.

Lactose, lactose derivatives, starch, starch derivatives, treated starch, chitin, cellulose and derivatives thereof, e.g. microcrystalline cellulose (e.g. Avicel), sucrose, dextrates, dextrin, dextrose, maltodextrin, hydrogenated vegetable oil, kaolin, alkali or alkaline earth salts such as calcium phosphates, e.g. dicalcium hydrogen phosphate (e.g. in the form of the dihydrate or preferably the anhydrate), calcium carbonate, magnesium carbonate, magnesium oxide, calcium sulphate, sodium chloride, potassium chloride and mixtures thereof can be used as fillers for the purposes of the invention. Similarly, SiO₂ modified microcrystalline cellulose (e.g. Prosolv®, Rettenmaier & Söhne, Germany) can be used.

Other fillers that can be used are sugar alcohols and/or sugars (especially monosaccharides and disaccharides) such as mannitol, sorbitol, xylitol, isomalt, glucose, fructose, maltose and mixtures thereof. In principle, it is also possible to use mixtures of the above-mentioned fillers.

The fillers are preferably selected from mannitol, microcrystalline cellulose, silicified microcrystalline cellulose, lactose, dicalcium hydrogen orthophosphate (preferably as the anhydrate) and starch.

Fillers are generally used in amounts between 10 and 99% by weight, preferably between 25 and 85% by weight, particularly preferably between 30 and 80% by weight, based on the total weight of the intermediate.

The intermediate of the invention preferably contains not only fillers, but also surface stabilisers (b₂). In general, surface stabilisers (b₂) are understood to mean substances which can prevent the reagglomeration of particles, especially milled particles. The surface stabiliser is preferably a polymer. In addition, the surface stabiliser also comprises substances which behave like polymers. Examples of these are fats and waxes. They also include low-molecular-weight oligomers, natural polymers or emulsifiers. Preferred surface stabilisers contain non-ionic or ionic emulsifiers.

The surface stabilisers (b₂) may be hydrophilic polymers. This refers to polymers which possess hydrophilic groups. Examples of suitable hydrophilic groups are hydroxy, amino, carboxy, sulphonate. In addition the hydrophilic polymer to be used for the preparation of the intermediate preferably has a weight-average molecular weight of 1,000 to 150,000 g/mol, more preferably from 2,000 to 90,000 g/mol. The weight-average molecular weight is preferably determined in the context of this application by means of gel permeation chromatography.

When the polymer used as the surface stabiliser (b₂) is dissolved in water in an amount of 2% by weight, the resulting solution preferably has a viscosity of 0.1 to 25 mPa·s, more preferably 1.0 to 18 mPa·s, especially 2 to 15 mPa·s, measured at 25° C. and determined in accordance with Ph. Eur., 6th edition, chapter 2.2.10. Especially in the case of HPMC, the resulting solution preferably has a viscosity of 2 to 10 mPa·s.

The intermediate of the invention may, for example, comprise the following hydrophilic polymers as the surface stabiliser: polysaccharides, such as hydroxypropyl methyl cellulose (HPMC), ethyl cellulose, methyl cellulose, hydroxyethyl cellulose, ethyl hydroxyethyl cellulose, hydroxypropyl cellulose (HPC), salts of carboxymethyl cellulose; polyvinyl pyrrolidone, polyvinyl acetate (PVAC), polyvinyl alcohol, polymers of acrylic acid and their salts, polyacrylamide, polymethacrylates, vinyl pyrrolidone/vinyl acetate copolymers (such as Kollidon® VA64, BASF), polyalkylene glycols and their derivatives, such as polypropylene glycol or preferably polyethylene glycol, polyethylene sorbitan fatty acid esters, co-block polymers of polyethylene glycol, especially co-block polymers of polyethylene glycol and polypropylene glycol (Pluronic®, BASF), and mixtures of the polymers mentioned.

The surface stabilisers preferably used are polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 45,000 to 75,000 g/mol and/or polymers of acrylic acid and their salts, especially with a weight-average molecular weight of 50,000 to 250,000 g/mol. In addition, HPMC is preferably used, especially with a weight-average molecular weight of 20,000 to 90,000 g/mol and/or preferably a proportion of methyl groups of 10 to 35% and a proportion of hydroxy groups of 1 to 35%. Likewise, HPC is preferably used, especially with a weight-average molecular weight of 50,000 to 100,000 g/mol. In addition, polyethylene glycol with a number-average molecular weight of 2,000 to 40,000 g/mol, especially 3,500 to 25,000 g/mol, is preferably used. Likewise, a polyethylene/polypropylene block copolymer is preferably used, wherein the polyethylene content is preferably 70 to 90% by weight. The polyethylene/polypropylene block copolymer preferably has a number-average molecular weight of 1,000 to 30,000 g/mol, more preferably from 3,000 to 15,000 g/mol.

Further examples of natural surface stabilisers are gelatine, casein, lecithin, dextran, gum arabic, gum tragacanth and/or cholesterol.

Fatty acids and their derivatives and salts, sorbitan esters and silicates can also be used. In the context of this invention, it is also possible to use any mixtures of the above-mentioned surface stabilisers.

Surfactants can likewise be used as surface stabilisers. It is preferable to use sodium lauryl sulphate (SDS).

In a preferred embodiment, a combination of polymeric surface stabiliser and surfactant is used, e.g. a combination of HPMC and SDS. The weight ratio of polymeric surface stabiliser to surfactant is preferably 50:1 to 3:1.

In the context of this invention, it has unexpectedly also been found that it is particularly advantageous especially to use surface stabilisers (b2) and/or fillers (b1) with low brittleness in order to produce the intermediate of the invention.

Excipients (especially surface stabilisers and fillers) can generally be classified with regard to the change in the shape of the particles under compression pressure (compaction): plastic excipients are characterised by plastic deformation, whereas when compressive force is exerted on brittle excipients, the particles tend to break into smaller particles. Brittle behaviour on the part of the surface stabiliser can be quantified by the increase in the surface area in a moulding. In the art, it is customary to classify brittleness in terms of the “yield pressure”. According to a simple classification, the values for the “yield pressure” here are low for plastic substances but high in the case of friable substances, on the other hand [Duberg, M., Nyström, C., 1982. Studies on direct compression of tablets VI. Evaluation of methods for the estimation of particle fragmentation during compaction. Acta Pharm. Suec. 19, 421-436; Humbert-Droz P., Mordier D., Doelker E. Méthode rapide de détermination du comportement à la compression pour des etudes de preformulation. Pharm. Acta Helv., 57, 136-143 (1982)]. The “yield pressure” describes the tension that has to be reached for the excipient (i.e. preferably the surface stabiliser and/or filler) to begin to flow plastically.

The “yield pressure” is preferably calculated using the reciprocal of the gradient of the Heckel plot, as described in York, P., Drug Dev. Ind. Pharm. 18, 677 (1992). The measurement in this case is preferably made at 25° C. and a deformation rate of 0.1 mm/s.

In the context of the present invention, an excipient (especially a surface stabiliser and/or filler) is deemed a non-brittle excipient if it has a “yield pressure value” of no more than 150 MPa, preferably 5 to 80 MPa. An excipient is usually described as a brittle excipient if it has a “yield pressure” of more than 80 MPa, preferably more than 150 MPa. Brittle excipients may have a yield pressure of up to 500 MPa.

Examples of preferred non-brittle fillers are mannitol or starch. Non-brittle substrates are preferably used in the preparation of the intermediate of the invention. Examples of brittle fillers are microcrystalline cellulose or dicalcium hydrogen phosphate. Brittle excipients, especially brittle fillers are preferably added in the granulation step (iii) and/or compressing step (iv) (described later on below). Alternatively, however, non-brittle fillers or a mixture of brittle and non-brittle fillers may be added in the granulation step (iii) and/or compressing step (iv).

Examples of preferred non-brittle excipients are HPMC and polyvinyl pyrrolidone, preferably with the above-mentioned molecular weights.

Surface stabilisers (b₂) are usually employed in amounts of 1 to 30% by weight, preferably 2 to 20% by weight, particularly preferably 3 to 15% by weight, based on the total weight of the intermediate.

Disintegrants (b₃) for the purposes of the invention are understood to mean substances which accelerate the disintegration of an oral dosage form, especially a tablet, after it is placed in water. Suitable disintegrants are, for example, organic disintegrants such as carrageenan, croscarmellose and crospovidone. Alkaline disintegrants are likewise used. The term “alkaline disintegrants” means disintegrants which, when dissolved in water, produce a pH level of more than 7.0. Croscarmellose or crospovidone are preferred.

Disintegrants are usually employed in amounts of 0 to 25% by weight, preferably 0.1 to 20% by weight, particularly preferably 3 to 15% by weight, based on the total weight of the intermediate.

In a further embodiment, the intermediate of the invention may additionally contain a flow conditioning agent (b₄).

The task of flow conditioning agents (b₄) is to reduce both the interparticular friction (cohesion) between the individual particles in a tableting mixture and their adherence to the wall surfaces of the press mould (adhesion). One example of an additive to improve the powder flowability is disperse silicon dioxide (e.g. Aerosil®). Preferably, silica is used with a specific surface area of 50 to 400 m²/g, determined by gas adsorption in accordance with Ph. Eur., 6th edition 2.9.26.

Flow conditioning agents are usually employed in amounts of 0 to 10% by weight, preferably 0.1 to 5% by weight, particularly preferably 1 to 3% by weight, based on the total weight of the intermediate.

In a further embodiment, the intermediate may additionally contain lubricants (b₅). Lubricants (b₅) are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate (Pruv®), magnesium stearate and/or calcium stearate.

Lubricants (b₅) are usually employed in amounts of 0 to 5% by weight, preferably 0.1 to 2% by weight, particularly preferably 0.5 to 1.5% by weight, based on the total weight of the intermediate.

The intermediate of the invention can contain components (a) and (b₁) to (b₅) explained above. In producing oral dosage forms, it is, however, optionally also possible that components (b₁), (b₃), (b₄) and (b₅) in particular are (at least partially) only added before or during the granulation and/or compressing steps (described below).

In a preferred embodiment, the intermediate of the invention contains

(a) entecavir between 0.1 and 10% by weight, preferably between 0.15 and 1.5% by weight, particularly preferably between 0.2 and 1% by weight,
(b₁) fillers between 5 and 90% by weight, preferably between 25 and 85% by weight, particularly preferably between 30 and 80% by weight,
(b₂) surface stabiliser between 1 and 30% by weight, preferably 2 to 20% by weight, particularly preferably 3 to 15% by weight,
(b₃) disintegrants between 0 and 40% by weight, preferably 1.0 to 20% by weight, particularly preferably 2.0 to 15% by weight,

Alternatively, the intermediate of the invention may also consist of the above-mentioned components (a) and (b₁) to (b₃).

The expression “total weight of the intermediate” refers in this context to the weight of the active agent and excipients contained in the intermediate. In other words, it refers to the weight of the intermediate without solvents (used, for example, in the wet milling process described below).

A further subject matter of the present invention is an oral dosage form containing the intermediate of the invention.

An oral dosage form for the purposes of the invention is understood to mean a drug formulation which is applied orally. Oral dosage forms in the context of this invention are preferably tablets or capsules. Alternatively, packages such as sachets or stickpacks containing the intermediate of the invention (optionally in granulated form) may also be regarded as oral dosage forms.

It is preferable for the oral dosage form of the invention to contain a substantial proportion of intermediate of the invention. The oral dosage form of the invention preferably contains more than 2 to 100% by weight intermediate of the invention, more preferably 5 to 80% by weight intermediate of the invention, even more preferably 10 to 50% by weight intermediate of the invention, particularly preferably 15 to 40% by weight, especially 20 to 35% by weight intermediate of the invention. This is based on the total weight of the active agent and excipients of the oral dosage form. This means, in the case of capsules, sachets or stickpacks for example, that the empty weight of the capsules, sachets or stickpacks is disregarded.

A further subject matter of the present invention relates to a process for preparing the intermediate of the invention. The production process of the invention comprises the steps of:
(i) mixing (a) entecavir and (b) excipients, and
(ii) grinding (a) entecavir and (b) excipients.

In step (i), (a) entecavir and excipients (b) are mixed.

“Mixing” is to be understood for the purposes of the invention as meaning a process of combining substances with the aim of achieving a substantially homogeneous distribution of different substances by the action of mechanical forces. Mixing for the purposes of the invention is performed in conventional mixing devices, such as roll mixers, shaking mixers, free-fall mixers, shear mixers, ploughshare mixers, planetary mixing kneaders, Z or sigma kneaders or fluid or intensive mixers. A free-fall mixer is preferably used.

The mixing time is usually 0.5 minutes to 1 hour, preferably 2 minutes to 50 minutes, more preferably 5 minutes to 30 minutes.

In step (i), (a) entecavir and excipients (b) are milled.

In general, “milling” is understood to mean the comminution of substances, especially active agents and excipients, to a predetermined particle size spectrum by applying an external force. The comminution principle can conventionally involve the effect of pressure, friction, cutting, impingement, impact, or combinations thereof.

Milling and grinding in the context of this invention can mean both wet grinding and also dry milling. According to the invention, “dry milling” is understood to mean the comminution of solids in the absence of solvents. According to the invention, “wet grinding” is understood to mean the comminution of solids in a liquid phase. The liquid phase here is preferably a liquid in which entecavir and excipients essentially do not dissolve. Examples of suitable grinding fluids are methanol, ethanol, isopropanol, acetone, chloroform, butanol, ethyl acetate, heptane, pentanol or mixtures thereof. Acetone is preferably used.

The milling is generally performed in conventional milling apparatuses, such as in a ball mill, air jet mill, pin mill, classifier mill, cross beater mill, disk mill, cutter mill, mortar grinder, rotor mill, roller crusher, hammer mill. It is preferable to use a peg mill, e.g. a Netsch MicroCer. The milling time is usually 0.5 minutes to 2 hours, preferably 2 minutes to 60 minutes, more preferably 5 minutes to 50 minutes.

In a preferred embodiment of this invention, the intermediate is produced by wet grinding followed by drying. For this purpose, spray-drying is preferably used as the drying step.

The milling and mixing conditions are generally selected such that an intermediate is obtained with the particle size distribution of the invention described above (D10, D50, D90, D90/D50, D50/D10, D90/D10). In particular, the milling temperature should be less than 220° C., preferably −178° C. to 180° C.

In addition, the mixing and milling conditions in the method of the invention are preferably selected such that an intermediate with a uniformity of the mixture of 90% to 110%, more preferably 92% to 108%, even more preferably 94% to 106%, particularly preferably 96% to 104% and especially 98% to 102%, is obtained.

The “uniformity of the mixture” refers here to the uniformity of the content of active agent in different intermediate samples. In order to determine the uniformity of the mixture in mixtures with a mass of less than 10 kg, five individual samples each are taken from the intermediate at random. In mixtures with a mass of 10 kg or more, ten individual samples are taken. The uniformity of the content of active agent is then determined in accordance with Ph. Eur. 6.0, Chapter 2.9.6, HPLC being used as the analytical process.

One subject matter of the invention is thus also an entecavir-containing intermediate in particulate form with a uniformity of the mixture of 90% to 110%, more preferably 92% to 108%, even more preferably 94% to 106%, particularly preferably 96% to 104% and especially 98% to 102%.

Steps (i) and (ii) can be performed in the process of the invention one after the other and/or simultaneously. The steps can optionally also be repeated. Hence, a cycle comprising mixing, grinding, mixing is possible. In a preferred embodiment, steps (i) and (ii) are performed according to the trituration method. In this process, entecavir (a) is first mixed and ground with part (preferably 10 to 50% by weight, particularly preferably 15 to 40% by weight) of the amount of excipients and after that, the remaining the amount of the excipients is added while grinding. The remaining the amount of the excipients is preferably added in one to 5 steps, especially in 2 to 4 steps.

In a further alternative embodiment, the invention relates to a process for preparing the oral dosage form, preferably a solid oral dosage form, containing the above-mentioned intermediate. One subject matter of the invention is thus a process comprising the steps
(i) mixing (a) entecavir and (b) excipients, and
(ii) grinding (a) entecavir and (b) excipients to produce an intermediate of the invention,
(iii) optionally granulating the intermediate,
(iv) pressing the intermediate from step (ii) or (iii) or filling the intermediate from step (ii) or (iii) into dosage forms such as sachets or capsules, wherein further pharmaceutical excipients may optionally be added before or during steps (iii) and (iv)

In step (i), (a) entecavir and excipients (b) are mixed. On this subject, reference is made to the preferred embodiments explained above.

In step (ii), (a) entecavir and excipients (b) are milled to produce an intermediate. On this subject, reference is made to the preferred embodiments explained above. Steps (i) and (ii) may, as explained above, be combined in any way desired.

In a preferred embodiment, further pharmaceutical excipients may optionally be added before or during steps (iii) and (iv).

In this context, it is possible for the excipients added before or during the granulation and/or compression step likewise to have the D10, D50 and D90 values for the particle size distribution explained above for the intermediate. It is, however, also possible for the excipients added before or during the granulation and/or compression step to have a larger (D50) particle size compared to the intermediate, e.g. a D50 value of 55 to 180 μm.

In a preferred embodiment, the excipients optionally added before or during the granulation and/or compression step have the following (D50) particle size:

fillers (b1) with a particle size (D50) from 55 to 180 μm, particularly preferably from 80 to 160 μm;
disintegrants (b3), flow conditioning agents (b4) and/or lubricants (b5) with a particle size (D50) of 1 to 100 μm, particularly preferably 5 to 50 μm.

Depending on the configuration of the process of the invention, various possibilities are conceivable for steps (iii) and (iv), e.g.:

Embodiment 1: Direct compression into tablets;
Embodiment 2: Dry granulation and subsequent compression into tablets;
Embodiment 3: Wet granulation and subsequent compression into tablets;
Embodiment 4: Dry granulation and subsequent filling into dosage forms such as sachets, stickpacks or capsules;
Embodiment 5: Wet granulation and subsequent filling into dosage forms such as sachets, stickpacks or capsules;
Embodiment 6: Spray drying and subsequent filling into dosage forms such as sachets, stickpacks or capsules;
Embodiment 7: spray-drying and subsequent compression into tablets;
Embodiment 8: Lyophilisation and subsequent filling into dosage forms such as sachets, stickpacks or capsules;
Embodiment 9: Lyophilisation and subsequent compression into tablets.

Embodiment 1 does not require a granulation step (iii), whereas embodiments 2 to 5 do. In the optional step (iii), the intermediate is therefore granulated.

“Granulating” is generally understood to mean the formation of relatively coarse or granular aggregate material as a powder by assembling and/or aggregating finer powder particles (agglomerate formation, or build-up granulation) and/or the formation of finer granules by breaking up coarser aggregates (disintegration, or break-down granulation). Granulation can conventionally mean wet or dry granulation. Dry granulation is generally carried out applying pressure or temperature. Wet granulation is generally carried out using surface stabilisers (b₂) and/or solvents. Granulation is generally carried out in conventional granulating devices, such as extruder, perforated-disk, perforated-roll, or fluidised-bed granulators. Compulsory mixers or spray dryers can likewise be used.

In the present application, the particle size distribution of granules is usually determined by means of screen analysis (differently from the method described above for intermediates and excipients (b)). Screening is performed in this connection for 10 minutes using appropriate screens in accordance with Ph. Eur 6.0 2.9.12., preferably with a Retsch® AS 200. The D50 value for the granule particle size distribution is the particle size at which 50% by weight of the particles have a smaller particle size than the particle size corresponding to the D50 value. Likewise, 50% by weight of the particles then have a larger particle size than the D50 value.

The granulation time, especially in the case of wet granulation, is usually 1 minute to 1 hour, preferably 2 minutes to 30 minutes. Dry granulation is usually carried out as a continuous process.

Preferred embodiments of dry and wet granulation will now be explained.

### Dry Granulation:

Dry granulation is usually preferred if the intermediate has been milled in a dry state.

In this embodiment of step (iii) of the method of the invention, the intermediate of the invention from step (ii) is compacted into a slug.

The compacting conditions in step (iii) are preferably selected such that the slug has a density of 1.03 to 1.8 g/cm³, especially 1.05 to 1.7 g/cm³.

The compacting is preferably carried out in a roll granulator.

The rolling force is preferably 2 to 50 kN/cm, more preferably 4 to 30 kN/cm, especially 10 to 25 kN/cm.

The gap width of the roll granulator is, for example, 0.8 to 5 mm, preferably 1 to 4 mm, more preferably 1.5 to 3 mm, especially 1.8 to 2.8 mm.

After that, the slug is preferably granulated. The granulation can be performed with methods known in the state of the art.

In a preferred embodiment, the dry granulation conditions are selected such that the resulting granules have a D50 value of 55 to 600 μm, more preferably 70 to 450 μm, even more preferably 100 to 400 μm, especially 150 to 350 μm.

In a preferred embodiment, the granulation of the slug is performed in a screen mill. In this case, the mesh width of the screen insert is usually 0.1 to 5 mm, preferably 0.4 to 3 mm, more preferably 0.61 to 2 mm, especially 0.7 to 1.8 mm.

For the dry granulation process, substantially only the intermediate of the invention is used. Optionally, but not preferably, smaller amounts of pharmaceutical excipients can be added which are not present in the particle size distribution of the invention. Examples of these are flow conditioning agents. In the dry granulation step, 90 to 100% by weight, more preferably 95 to 99.9% by weight, of the intermediate of the invention is preferably used, based on the total weight of the substances used.

### Wet Granulation:

Wet granulation can be performed with conventional methods. Wet granulation is usually preferred if the intermediate of the invention has been produced by means of a wet grinding process. Wet granulation is preferably carried out in a fluidised bed.

For this purpose, the intermediate from step (ii), preferably the moist intermediate from step (ii), is introduced into a fluidised bed. In a preferred embodiment, substrate cores can be prepared in advance in the fluidised bed.

Possible materials for substrate cores are, for example, the above-mentioned fillers. It is preferable to use microcrystalline cellulose. The substrate cores usually have a particle size (D50 value) of 55 to 500 μm, more preferably 60 to 350 μm, even more preferably 90 to 250 μm, especially 110 to 220 μm. The weight ratio of intermediate of the invention to substrate cores is preferably 10:1 to 1:2, more preferably 5:1 to 2:1. The weight ratio specified here preferably refers to the dry intermediate.

For the wet granulation process, substantially only the intermediate of the invention and optionally the substrate cores explained above are used. Optionally, but not preferably, smaller amounts of further pharmaceutical excipients may be added. In the wet granulation step, 30 to 100% by weight, more preferably 60 to 99.9% by weight, even more preferably 70 to 99.0% by weight, of the intermediate of the invention is preferably used, based on the total weight of the substances used.

In a preferred embodiment, the wet granulation is carried out in a fluidised bed granulator, such as a Glatt® GPCG 3 (Glatt GmbH, Germany).

If in steps (ii) or (iii) the basic operations of wet granulation and/or wet milling are performed, it is normal to carry out a step of “drying”. The drying step can be performed after or at the same time as the granulation step.

“Drying” for the purposes of this invention is understood to mean the separation of liquids adhering to solids. The adhering liquids are preferably water in the form of contact moisture, capillary water, hydration water, adsorption water, and water of constitution. Drying generally takes place in conventional drying equipment, such as cabinet or tray dryers, vacuum dryers, fluidised bed dryers, spray dryers or freeze dryers. The drying and granulation process is preferably performed in one and the same apparatus.

The drying conditions are preferably selected such that the content of water in the resulting granules is 0.1 to 5% by weight. The content of residual solvent is preferably 1 to 1,000 ppm, preferably 5 to 500 ppm.

In a preferred embodiment, the wet granulation conditions are selected such that the resulting particles (granules) have a particle size (D50 value) of 55 to 600 μm, more preferably 70 to 450 μm, even more preferably 100 to 400 μm, especially 150 to 350 μm.

In addition, the granulation conditions in all the granulation processes are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.03 to 1.3, more preferably from 1.04 to 1.20 and especially from 1.04 to 1.15. The “Hausner factor” in this context means the ratio of tapped density to bulk density. The bulk density and tapped density are determined in accordance with USP 24, test 616 “Bulk Density and Tapped Density”.

In addition, the mixing, milling and/or granulation conditions are preferably selected such that granules with a uniformity of the mixture of 90% to 110%, more preferably 92% to 108%, even more preferably 94% to 106%, particularly preferably 96% to 104% and especially 98% to 102%, are obtained.

The “uniformity of the mixture” refers here to the uniformity of the content of active agent in different granule samples. In order to determine the uniformity of the mixture of the granules with a mass of less than 10 kg, five individual samples each are taken from the granules at random, and the uniformity of the content of active agent is determined as explained above. In granule mixtures with a mass of 10 kg or more, ten individual samples are taken.

Another subject matter of the invention is thus granules obtainable by a process comprising the steps of
(i) mixing (a) entecavir and (b) excipients, and
(ii) grinding (a) entecavir and (b) excipients to produce an intermediate of the invention,
(iii) granulating the intermediate, wherein further pharmaceutical excipients are optionally added before or during the granulation step.

Hence, yet another subject matter of the invention is entecavir-containing granules with a uniformity of the mixture of 90% to 110%, more preferably 92% to 108%, even more preferably 94% to 106%, particularly preferably 96% to 104% and especially 98% to 102%. The granules of the invention can also be filled into capsules, sachets or stickpacks, for example, for application as an oral dosage form.

In a preferred embodiment of the present invention, in step (iv) the intermediate from step (ii) or the granulated intermediate from step (iii) are compressed into tablets.

The process of compressing can be carried out, as explained above, without further pre-treatment by compressing the intermediate from step (ii) (=direct compression) or after the granulation carried out in step (iii). Direct compression is preferred.

For the compressing step, substantially only the intermediate of the invention—optionally in granulated form as described above—is used. Optionally, but not preferably, smaller amounts of pharmaceutical excipients can be added which are not present in the particle size distribution of the invention. Examples of these are flow conditioning agents, disintegrants and lubricants. In the compressing step, 60 to 100% by weight, more preferably 70 to 99.9% by weight intermediate of the invention or 80 to 100% by weight, more preferably 85 to 99.9% by weight, granules resulting from process step (iii) are preferably used, based on the total weight of the compressed substances.

The tableting conditions here are preferably selected such that the resulting tablets have a ratio of tablet height to weight of 0.005 to 0.3 mm/mg, particularly preferably 0.05 to 0.2 mm/mg.

Conventional tableting machines used in the production of tablets can be employed. Rotary presses or eccentric presses are preferably used. In the case of rotary presses, a compressive force of 2 to 40 kN, preferably 2.5 to 35 kN, is usually applied. In the case of eccentric presses, a compressive force of 1 to 20 kN, preferably 2.5 to 10 kN, is usually applied. By way of example, the Korsch® EKO is used.

According to the invention, the resulting tablets preferably have a mass of 100 to 550 mg, preferably 150 to 450 mg, particularly preferably 180 to 420 mg, based on the total weight of the non-film-coated tablet. The resulting tablets preferably have a mass of 350 to 450 mg, particularly preferably 370 to 430 mg per mg entecavir.

In a preferred embodiment, the oral dosage form of the invention, especially the tablet of the invention, contains

(a) entecavir between 0.1 and 2% by weight, preferably between 0.2 and 1.5% by weight, particularly preferably between 0.4 and 1.2% by weight,
(b₁) fillers between 30 and 99.8% by weight, preferably between 55 and 98% by weight, particularly preferably between 75 and 95% by weight,
(b₂) surface stabiliser between 0.1 and 30% by weight, preferably 0.5 to 15% by weight, particularly preferably 1.0 to 10% by weight,
(b₃) disintegrant between 0 and 30% by weight, preferably 1.0 to 20% by weight, particularly preferably 3.0 to 15% by weight,
(b₄) flow conditioning agent between 0 and 10% by weight, preferably 0.1 to 6.0% by weight, particularly preferably 0.8 to 4.0% by weight,
(b₅) lubricants between 0 and 10% by weight, preferably 0.1 to 5% by weight, particularly preferably 0.5 to 3.0% by weight, based on the total weight of the non-film-coated tablet.

In the context of the invention, the resulting tablets may be coated or uncoated. In accordance with the invention, the film formers used for the coating process may preferably be cellulose derivatives, such as methyl cellulose (MC), ethyl cellulose (EC), hydroxyethyl cellulose (HEC), methacrylic acid/acrylate copolymers, such as methacrylic acid/methacrylate copolymer or methacrylic acid/methyl methacrylate copolymer, vinyl polymers, such as polyvinyl pyrrolidone or polyvinyl acetate phthalate or natural film formers, such as shellack.

The thickness of the coating is usually 0.1 to 100 μm, preferably 1 to 80 μm.

It is preferable for the optionally applied film to have substantially no effect on the release. These are therefore preferably films with no influence on the release of the active agent. In the context of this invention, it is preferable for neither enteric film coatings nor delayed-release coatings to be used.

For the purposes of the invention, the resulting tablets should preferably exhibit a high level of hardness and low friability.

The resulting tablets preferably have a hardness of 50 to 300 N, particularly preferably 80 to 250 N, especially 100 to 220 N. The hardness is determined in accordance with Ph. Eur. 6.0, section 2.9.8.

In addition, the resulting tablets preferably have a friability of 0.1 to 0.8%, preferably 0.2 to 0.6% and particularly preferably 0.3 to 0.5%. The friability is determined in accordance with Ph. Eur. 6.0, section 2.9.7.

The release profile of the tablets of the invention according to the USP method (paddle, 900 ml test medium in phosphate buffer at pH 6.8 and 37° C., 75 rpm) after 10 minutes usually indicates a content released of at least 30%, preferably at least 50%, especially at least 70%.

In a further embodiment of the present invention, in step (iv) the intermediate from step (ii) or the granules from step (iii) are filled into dosage forms such as sachets, stickpacks or capsules.

The invention will now be explained with reference to the following examples.

### EXAMPLES

### A) Preparation of the Intermediate/Oral Dosages Forms of the Inventions

Four different intermediates in accordance with the invention containing entecavir (Examples 1 to 4) and a conventional powder mixture containing entecavir (Comparative example 1) were prepared.

In addition, four tablets of the invention containing entecavir in accordance with Examples 1 to 4 and conventional tablets containing entecavir in accordance with Comparative example 1 were prepared.

### Example 1

### Formulation

| | | |
|---|---|---|
| Entecavir (monohydrate) | 1 | mg |
| Mannitol | 70 | mg |
| Microcrystalline cellulose (MCC) | 150 | mg |
| Hydroxypropyl methyl cellulose (HPMC) | 15 | mg |
| Sodium carboxymethyl cellulose | 10 | mg |
| Colloidal silica (Aerosil ®) | 4 | mg |
| Magnesium stearate | 2 | mg |

### Intermediate:

Entecavir was ground in a mortar for 5 min. together with 15 mg mannitol. After that, HPMC was added and ground again for 5 minutes. In two further steps, half of the remaining mannitol was added and ground again for 5 minutes in each case.

### Tablet:

The resulting intermediate was mixed with MCC, sodium carboxymethyl cellulose and Aerosil® for 20 minutes in a free-fall mixer (Turbula® TB10), magnesium stearate was added through a 0.5 mm screen, and the finished mixture was mixed for a further 3 min.

The mixture produced was compressed into tablets on a Korsch eccentric press.

### Example 2

### Formulation

| | | |
|---|---|---|
| Entecavir (monohydrate) | 0.5 | mg |
| Corn starch | 70 | mg |
| Dicalcium hydrogen phosphate (Dicafos) | 150 | mg |
| Hydroxypropyl methyl cellulose (HPMC) | 15 | mg |
| Sodium carboxymethyl cellulose | 10 | mg |
| Colloidal silica (Aerosil ®) | 4 | mg |
| Magnesium stearate | 2 | mg |

### Intermediate:

Entecavir was ground in a mortar for 5 min. together with 15 mg corn starch. After that, HPMC was added and ground again for 5 minutes. In two further steps, half of the remaining corn starch was added and ground again for 5 minutes in each case.

### Tablet:

The resulting intermediate was mixed with Dicafos, sodium carboxymethyl cellulose and Aerosil® for 20 minutes in a free-fall mixer (Turbula® TB10). The resulting mixture, with 2 mg magnesium stearate added through a 0.5 mm screen followed by 3 minutes of mixing in the free-fall mixer, was compressed into tablets on a Korsch eccentric press.

### Example 3

### Formulation

| | | |
|---|---|---|
| Entecavir (monohydrate) | 1 | mg |
| Lactose monohydrate | 160 | mg |
| Starch | 80 | mg |
| Hydroxypropyl methyl cellulose (HPMC) | 10 | mg |
| Colloidal silica (Aerosil ®) | 4 | mg |
| Cross-linked polyvinyl pyrrolidone | 8 | mg |
| Magnesium stearate | 2 | mg |

### Intermediate:

Entecavir and lactose monohydrate were present in micronised form (D50<50 μm). The two substances were mixed step by step.

### Tablet:

The intermediate and the further excipients (with the exception of the magnesium stearate) were mixed for 30 minutes in a free-fall mixer (Turbula® TB10). The mixture, with 2 mg magnesium stearate added through a 0.5 mm screen followed by 3 minutes of mixing in the free-fall mixer, was compressed into tablets on a Korsch eccentric press.

### Example 4

### Formulation

| | | |
|---|---|---|
| Entecavir (monohydrate) | 1 | mg |
| Hydroxypropyl methyl cellulose (HPMC) | 1 | mg |
| SDS | 0.1 | mg |
| Microcrystalline cellulose | 20 | mg |
| Silicified microcrystalline cellulose | 200 | mg |
| Colloidal silica (Aerosil ®) | 4 | mg |
| Sodium carboxymethyl cellulose | 10 | mg |
| Magnesium stearate | 2 | mg |

### Intermediate:

Entecavir was ground in acetone for 1 h, together with HPMC and SDS, in a Netzsch® MicroCer.

Microcrystalline cellulose (D50 approx. 100 μm) was added to the ground suspension containing the intermediate of the invention, and this was spray-dried on a Büchi® spray tower.

### Tablet:

After the spray drying, the mixture consisting of MCC and intermediate was mixed for 25 min, together with the remaining excipients except for magnesium stearate, in a free-fall mixer (Turbula® TB10). After that 2 mg magnesium stearate was added through a 0.5 mm screen and mixed again for 3 min. The resulting mixture was compressed into tablets on a Korsch eccentric press.

### Comparative Example 1

The same formulation was used as in Example 1. As excipients and active agent (entecavir), non-micronised, standard commercial products were used, without grinding:

Entecavir was mixed together with 15 mg mannitol for 5 minutes in a free-fall mixer (Turbula® TB10). After that, HPMC was added and mixed again for a further 5 minutes. In two further steps, half of the remaining mannitol was added and mixed again for 5 minutes in each case.

The D50 value for the particle size distribution of the powder mixture was outside the range claimed according to the invention.

The resulting intermediate was mixed with MCC, sodium carboxymethyl cellulose and Aerosil® for 20 minutes in the free-fall mixer (Turbula® TB10), magnesium stearate was added through a 0.5 mm screen, and the finished mixture was mixed for a further 3 min.

The mixture produced was compressed into tablets on a Korsch eccentric press.

### B) Physical Properties

### 1. Content Uniformity

A content uniformity test in accordance with Ph. Eur. 6.0 section 2.9.6 was performed on the tablets prepared in Examples 1 to 4 and Comparative example 1. In that test, 10 units were investigated and checked to see whether each individual content of entecavir in those 10 units lay between 85 and 115 percent of the average content. The content of entecavir was determined by means of HPLC.

The content of entecavir in the tablets prepared according to Examples 1 to 4 exhibited a content uniformity in 10 units each of between 91% and 109% of the average content. The tablets of the invention prepared according to Examples 1b to 3b therefore passed the content uniformity test.

In contrast to this, the tablets prepared according to Comparative example 1 exhibited a content uniformity in 9 units of between 86% and 113%. One tablet exhibited a content uniformity of 117% of the average content, whereupon 20 further tablets were tested for content uniformity in accordance with Ph. Eur. 6.0 section 2.9.6. 29 of the tablets tested exhibited a content within the limits of 85% and 115% of the average content of the 30 tablets. The content of one tablet exhibited a content within the limits of 75% and 125%. The tablets prepared according to the Comparative examples therefore passed the content uniformity test.

### Intoxication/Ensuring Sufficient Bioavailability

The tablets prepared in accordance with Examples 1 to 4 exhibited a sufficiently high level of bioavailability, with no indications of intoxication occurring.

1. An intermediate comprising (a) entecavir and (b) one or more excipients, wherein the D50 value for the particle size distribution of the intermediate is less than about 50 μm.

2. The intermediate of claim 1, wherein the particle size distribution of the intermediate is monomodal.

3. The intermediate of claim 1, wherein the one or more excipients are selected from (b₁) fillers, (b₂) surface stabiliser, and/or (b₃) disintegrants.

4. The intermediate of claim 1, comprising
(a) entecavir between about 0.1 and about 10% by weight,
(b₁) a filler between about 40 and about 98.9% by weight,
(b₂) a surface stabiliser between about 1.0 and about 25% by weight,
(b₃) a disintegrant between about 0 and about 30% by weight,
based on the total weight of the intermediate.

5. The intermediate of claim 1, wherein the D10 value for the particle size distribution of the intermediate is between about 4.0 and about 12 μm.

6. The intermediate of claim 1, wherein the D90 value for the particle size distribution of the intermediate is less than about 200 μm.

7. The intermediate of claim 1, wherein the ratio between the D90 value and D50 value D90/D50 is between about 2 and about 7.

8. The intermediate of claim 1, wherein the D50 value for the particle size distribution of the intermediate is from about 1 to about 20 μm.

9. An oral dosage form comprising an intermediate of claim 1.

10. The oral dosage form of claim 9, wherein the uniformity of the content of entecavir, determined in accordance with Pharm. Eur. 2.9.6, in the first ten units is characterised such that each individual content of entecavir is between about 90 and about 110 percent of the average content.

11. The oral dosage form of claim 10, wherein the oral dosage form is in a tablet form.

12. A process for preparing an intermediate comprising (a) entecavir and (b) one or more excipients, wherein the D50 value for the particle size distribution of the intermediate is less than about 50 μm, comprising the steps of
(i) mixing (a) entecavir and (b) the one or more excipients, and
(ii) grinding the mixture of (a) entecavir and (b) the one or more excipients.

13. A process for preparing an oral dosage form, comprising the steps of
(i) mixing (a) entecavir and (b) one or more excipients, and
(ii) grinding the mixture of (a) entecavir and (b) the one or more excipients to produce an intermediate wherein the D50 value for the particle size distribution of the intermediate is less than about 50 μm,
(iii) optionally granulating the intermediate,
(iv) compressing the intermediate from step (ii) or (iii) into tablets or filling the intermediate from step (iii) into dosage forms, preferably sachets or capsules,
wherein one or more further pharmaceutical excipients are optionally added before or during steps (iii) and (iv).

14. Granules prepared by a process comprising the steps of
(i) mixing (a) entecavir and (b) one or more excipients, and
(ii) grinding the mixture of (a) entecavir and (b) the one or more excipients to produce an intermediate wherein the D50 value for the particle size distribution of the intermediate is less than about 50 μm,
(iii) granulating the intermediate,
wherein one or more further pharmaceutical excipients are optionally added before or during the granulation step.

15. The granules of claim 14 with a uniformity of the mixture of about 90 to about 110%.

16. Micronised entecavir.

17. Micronised entecavir for the treatment of chronic hepatitis B virus infections, wherein HBeAg-negative patients are treated.
